(19) United States
(12) Patent Application Publication
(10) Pub. No.: US 2024/0018554 A1
(43) Pub. Date: Jan. 18, 2024

(54) HIGH VALUE PROTEIN PRODUCTION FROM WASTE ORGANIC STREAMS AT AN EXISTING ETHANOL PLANT

(71) Applicant: LE3 Technology LLC, De Soto, KS (US)

(72) Inventor: Scott A. Lucas, De Soto, KS (US)

(21) Appl. No.: 18/223,346

(22) Filed: Jul. 18, 2023

Related U.S. Application Data
(60) Provisional application No. 63/390,181, filed on Jul. 18, 2022.

(51) Int. Cl. C12P 7/14; C12M 1/00

(52) U.S. Cl. CPC C12P 7/14; C12M 23/58; C12R 2001/84

(57) ABSTRACT

A system for converting waste organic material from an ethanol plant to high-protein, single-cell organism matter. The system includes first and second fermenters receiving waste organic material streams. A sterilizer is connected to the fermenters and is configured for sterilizing the output thereof.

### CROSS-REFERENCE TO RELATED APPLICATION

This application claims priority in U.S. Provisional Patent Application No. 63/390,181, filed Jul. 18, 2022, which is incorporated herein by reference.

### BACKGROUND OF THE INVENTION

### 1. Field of the Invention

The present invention relates generally to a system and method for converting waste organic streams produced at ethanol plants to a high protein product.

These waste organic streams can be described as thin stillage from the existing ethanol plant or cellulosic hydrolysate that is produced from converting corn fiber from the existing plant using additional technology described in a separate patent application.

### 2. Description of the Related Art

Systems and methods have previously been available for converting waste organic streams to a high protein product. However, none has been available with the advantages and features of the present invention.

### BRIEF SUMMARY OF THE INVENTION

This invention generally provides a system and method for an aerobic fermentation system that will be used to convert waste organic streams to a high protein product using additional technology covered by separate patent applications.

### BRIEF DESCRIPTION OF THE DRAWINGS

The drawings constitute a part of this specification and include exemplary embodiments of the present invention and illustrate various objects and features thereof.

FIG. 1 is a schematic diagram of an aerobic fermentation system embodying an aspect of the present invention.

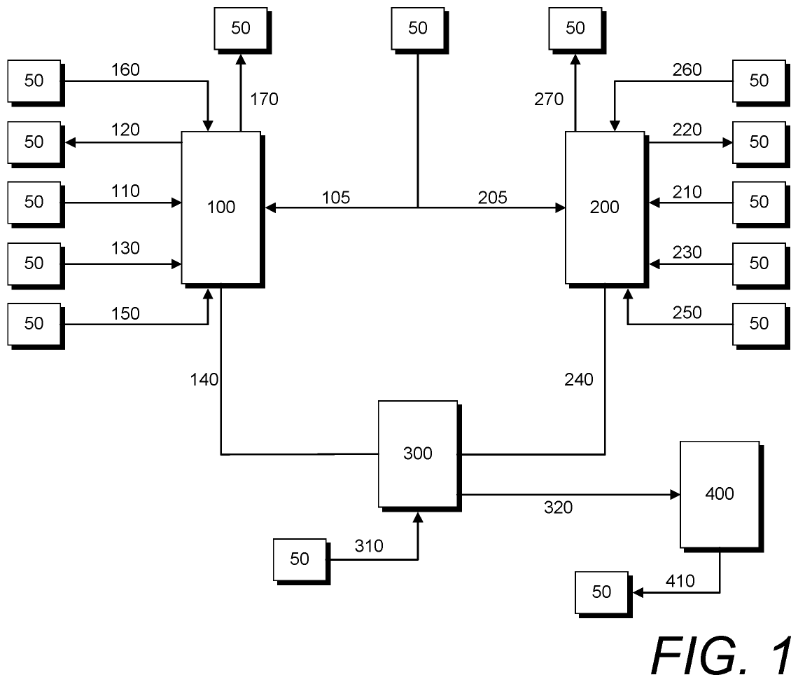

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. Said terminology will include the words specifically mentioned, derivatives thereof and words of similar meaning.

### II. Preferred Embodiment Aerobic Fermentation System

Waste organic material, 105 and 205, will be fed to two separate aerobic fermenters, 100 and 200, on a continuous basis. Each fermenter will be agitated.

An appropriate organism, 160 and 260, such as Torula (Cyberlindnera jadinii) or Pichia pastoris will be utilized to convert the waste organic material to single cell organism matter which will be high in protein. These organisms will be fed to the fermenters at the start of the new batch and allowed to grow until the growth rate is no longer productive and then the fermenter will be emptied.

The fermenters will be run in parallel with each fermenter taken out of service and cleaned on a periodic basis. Each fermenter will be in operation for a period of 24 to 60 hours depending on fermenter performance before the fermenter is emptied, cleaned, sterilized and put back in service.

Each fermenter will be cooled using jackets installed on the outside of the vessel and cooled using chilled water supply, 110 and 210, with chilled water return, 120 and 220, sent back to the chiller system.

Each fermenter will be fed sterile air, 150 and 250, at an appropriate rate to help facilitate the optimum growth of the organisms.

Air and exhaust gas, 170 and 270, from the organism growth will be vented back to the existing ethanol plant 50 to be processed.

The contents of each fermenter will be pulled from the fermenter on a continuous basis and passed through a sterilizer, 300, that will heat the stream to approximately 190 F using steam, 310, and passed along to the Broth Surge Tank, 400 as heated broth, 320.

Fermenter broth, 410, will be sent back to the existing ethanol plant where it will be processed using separate technology.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A system for converting waste organic material streams to a high value protein product, which system comprises:
an ethanol plant configured for providing an output consisting of first and second waste organic material streams;
first and second aerobic fermenter vessels each receiving a respective waste organic material stream;
each said fermenter configured for utilizing an organism for converting waste organic material to high-protein, single-cell organism matter;
a water chiller system;
each said fermenter vessel having a cooling jacket configured for receiving a chilled water flow from said water chiller system and returning said chilled water via a chilled water return to the chiller system;
each said fermenter configured for receiving sterile air at an appropriate rate to facilitate organism growth;
each said fermenter configured for venting air and exhaust gas from the organism growth therein to the ethanol plant;
a sterilizer configured for receiving the contents of said fermenters;
a broth surge tank configured for receiving the contents of said fermenters as heated broth; and
said system configured for returning said fermenter brought to said ethanol plant.

2. The system according to claim 1, wherein at least one of said fermenters contains Torula (Cyberlindnera jadinii).

3. The system according to claim 1, wherein at least one of said fermenters contains Pichia pastoris.
